(19) United States
(12) Patent Application Publication
(10) Pub. No.: US 2014/0012334 A1
(43) Pub. Date: Jan. 9, 2014

(54) MUTIPLE ZONE BONE FASTENER

(71) Applicants: Rex W. Armstrong, Cordova, TN (US); Rodney R. Ballard, Lakeland, TN (US)

(72) Inventors: Rex W. Armstrong, Cordova, TN (US); Rodney R. Ballard, Lakeland, TN (US)

(73) Assignee: WARSAW ORTHOPEDIC, INC., Warsaw, IN (US)

(21) Appl. No.: 13/540,909

(22) Filed: Jul. 3, 2012

(51) Int. Cl. A61B 17/86

(52) U.S. Cl. 606/309; 606/312

(57) ABSTRACT

A multiple zone bone fastener includes a head attached to an elongated shaft with a leading edge. The elongated shaft has a first zone, a second zone and a third zone, such that the second zone is disposed between first and third zones. The shaft has a continuous helical thread along the length of the first, second and third zones wherein the distance between the threads in the second zone is twice the distance between the threads in the first and second zones.

### TECHNICAL FIELD

The present disclosure generally relates to medical devices for the treatment of bone disorders, and more particularly to a bone fastener configured for fixation into bone, such as the pelvis, having distinct regions with differing characteristics.

### BACKGROUND

Bone in humans and other mammals is generally classified into cortical bone, also known as lamellar or compact bone, and cancellous bone, also known as trabecular or spongy bone. Cortical bone and cancellous bone are classified based on porosity. Cortical bone is very dense, having a porosity ranging between 5% and 10%, while cancellous bone is much more porous with porosity ranging between 50% to 90%. Certain bones, such as the pelvic bone, for example, consist of cancellous bone between layers of cortical bone.

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders include stabilization of the region using implants and bone fasteners. This disclosure describes improved bone fasteners for use in surgical treatment of spinal disorders and spinal injuries.

### SUMMARY OF THE INVENTION

Accordingly, a bone fastener having a continuous helical thread and three separate zones is provided. The zones are configured to have a different distance between threads in each region. The distance between threads is specifically configured so as to maximize attachment to the region of bone they are designed to be fixed within. For example, since cortical bone is denser than cancellous bone, the first and third zones are designed to thread into cortical bone. That is, these zones have threads that are closer together than threads in the second zone, which is designed to thread into bone that is less dense than cortical bone such as cancellous bone.

In one embodiment, in accordance with the principles of the present disclosure, a bone fastener comprises a head attached to an elongated shaft with a leading edge. The elongated shaft has a first zone, a second zone and a third zone, such that the second zone is disposed between first and third zones. The shaft having a continuous helical thread along the length of the first, second and third zones wherein the distance between the threads in the second zone is twice the distance between the threads in the first and second zones.

In one embodiment, a bone fastener specifically configured for anchoring into a bone of a patient having cancellous bone between a first and second layer of cortical bone. The bone fastener having a head and an elongated shaft with a leading edge. The shaft of the bone fastener having a first zone, a second zone and a third zone, such that the second zone is disposed between the first and third zones. The shaft having one continuous helical thread along the length of the first, second zone and third zones such that the distance between threads in the second zone is twice a distance between threads in the first and third zones so that the first and third zones are configured to engage cortical bone and the second zone is configured to engage cancellous bone when the bone fastener is fully set within the bone of the patient.

The first and the second zones of the bone fastener are configured to have more threads, for example, twice as many threads, in the first and third zones that are configured to get into denser cortical bone and fewer threads in the second zone when set in the cancellous bone positioned between two layers of cortical bone, such as in the pelvis of a patient. The fewer threads in the second zone are configured to have more bone between the threads so as to be better set into the cancellous bone.

In yet another embodiment in accordance with the principles of the present disclosure, a method of fixation of the bone fastener of the present disclosure to a bony body is provided which includes the steps of exposing a surface of the bony body to receive the bone fastener and threading the self-tapping lead portion of the bone fastener into the bony body. The self-tapping lead portion of the bone fastener configured to cut a helical groove in the first layer of cortical bone, followed by the cancellous portion and then in the second layer of cortical bone in which the first zone of the bone fastener will reside. When set in a bone having cancellous bone between a first and second layer of cortical bone, such as the pelvis, the first zone will set in the first layer of cortical bone, the second zone will set in the cancellous bone and the third zone will set in the second layer of the cortical bone.

### BRIEF DESCRIPTION OF THE DRAWINGS

The present disclosure will become more readily apparent from the specific description accompanied by the following drawings, in which:

FIG. 1 is a side view of one particular embodiment of a bone fastener in accordance with the principles of the present disclosure; and

FIG. 2 is a side view of the bone fastener shown in FIG. 1 implanted in a bone.

Like reference numerals indicate similar parts throughout the figures.

### DETAILED DESCRIPTION OF THE INVENTION

The exemplary embodiments of bone fastener are discussed in terms of medical devices for the treatment of bone disorders and more particularly, in terms of a bone fastener having a continuous thread specifically configured to set into bone having cancellous bone between a first and second layer of cortical bone, such as the pelvis. In particular, the present disclosure is directed to a bone fastener having three zones wherein the distance between threads in the second zone is approximately twice the distance between threads in the first and third zones. The bone fastener in accordance with the principles of the disclosure is configured so as to provide additional threads for setting in the dense cortical bone in the first and third zones and fewer threads, therefore more bone between threads, to set into the cancellous bone in the second zone. Having more bone between threads in the porous cancellous area and more threads in the dense cortical bone provides for a secure placement of the bone fastener of the present disclosure.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft, implantable prosthetics and/or dynamic stabilization applications. It is contemplated that the present disclosure may be employed with other osteal and bone related applications, including those associated with diagnostics and therapeutics. It is further contemplated that the disclosed bone fastener may be employed in a surgical treatment with a patient in a prone or supine position, employing a posterior, lateral or anterior approach. Although the bone fastener of the present disclosure may be employed with procedures for treating the lumbar, cervical, and thoracic regions, it is specifically designed for bone having cancellous bone between a first and second layer of cortical bone, such as the pelvis.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The components of the bone fastener of the present disclosure are fabricated from materials suitable for medical applications, including metals, polymers, ceramics, biocompatible materials and/or their composites, depending on the particular application and/or preference of a medical practitioner. For example, the bone fastener can be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™ manufactured by Biologix Inc.), rigid polymers including polyphenylene, polyamide, polyimide, polyetherimide, polyethylene, polyurethanes of any durometer, epoxy and silicone. The bone fastener may also be fabricated from a heterogeneous material such as a combination of two or more of the above-described materials to achieve various desired characteristics such as strength, rigidity, elasticity, compliance, biomechanical performance, durability and radiolucency or imaging preference.

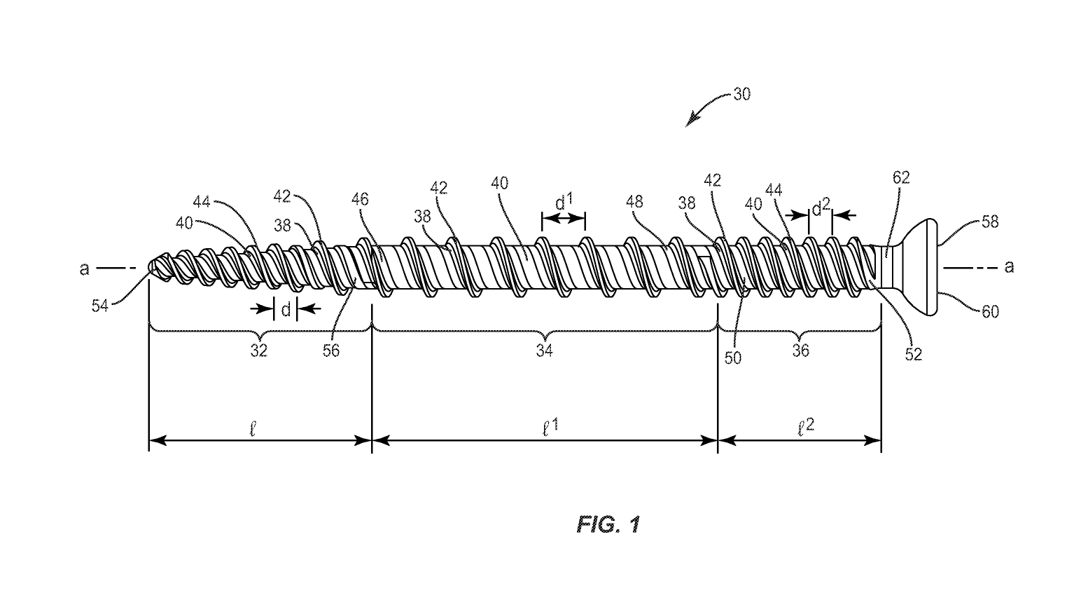

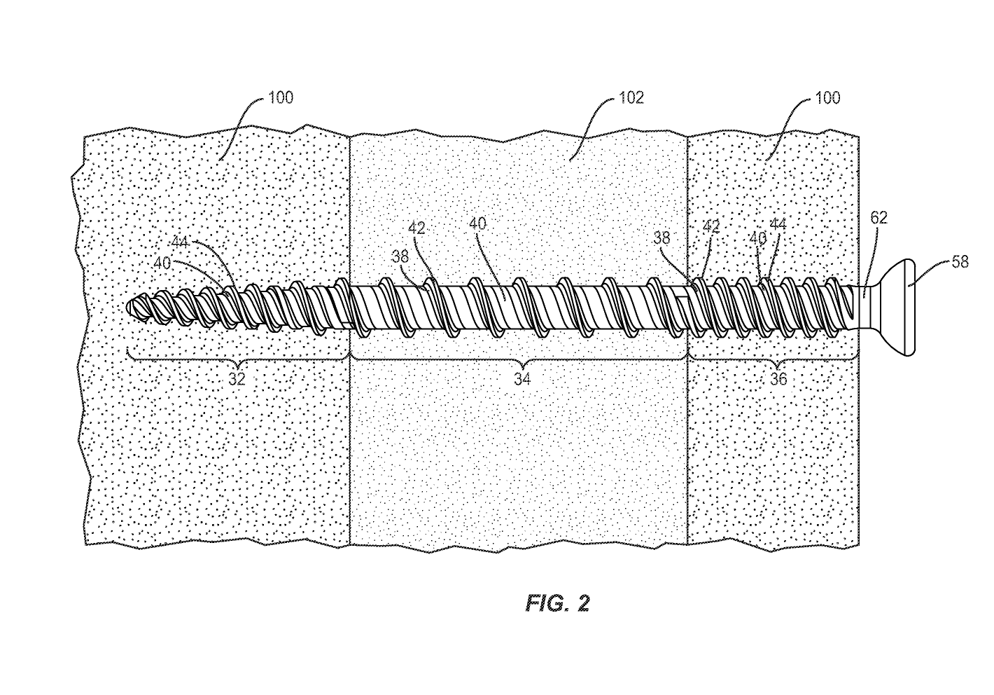

The following discussion includes a description of a bone fastener and related components in accordance with the principles of the present disclosure. Alternate embodiments are also disclosed. Reference will now be made in detail to the exemplary embodiments of the present disclosure, which are illustrated in the accompanying figures. Turning now to FIGS. 1-2, there is illustrated components of a dual lead bone fastener 30 in accordance with the principles of the present disclosure. It is envisioned that the bone fastener 30 in accordance with the principles of the present disclosure can also have triple, quadruple, or any other number of thread leads so as to achieve the same recited goals of the dual lead bone fastener 30 discussed further herein.

Bone fastener 30 is configured for fixation within bone, such as, for example, the pelvis, during surgical treatment of a spinal disorder, examples of which are discussed herein. Bone fastener 30 includes an elongated shaft having a cylindrical cross section with a leading edge 54 at one end and a head 60 at the opposite end disposed along a longitudinal axis a. The elongated shaft having a first zone 32, a third zone 36, and a second zone 34 positioned between the first and third zones 32, 36. The first and third zones 32, 36 are configured to be fixed within cortical bone and the second zone 34 is configured to be fixed within cancellous bone. It is envisioned that bone fastener 30 may be monolithically formed such that first zone 32, second zone 34 and third zone 36 are continuously formed with one another so as to prevent one zone from rotating differently relative to any other zone. First zone 32 has a length l extending between a distal end 54 and a proximal end 56 of first zone 32. Intermediate portion 34 has a length l¹ extending between a distal end 46 and a proximal end 48 of the second zone 34 wherein the proximal end 56 of the first zone 32 is continuous with the distal end 46 of the second zone 34 . The third zone 36 has a length l² extending between a distal end 50 and a proximal end 52 wherein the proximal end 48 of second zone 34 is continuous with the distal end 50 of the third zone 36. In one embodiment, length l and length l² are approximately equivalent and are both less than length l¹. However, it is envisioned that length l may be greater than length l², or vice versa. It is contemplated that the first zone 32, second zone 34 and the third zone 36 can have different dimensions, for example, with regard to length, width, diameter and thickness, according to the bone in which it is designed to be set into. In one embodiment, bone fastener 30 has a longitudinal bore extending through the length of bone fastener 30 such that bone fastener 30 is cannulated along axis a.

In one embodiment, in accordance with the principles of the present disclosure, bone fastener 30 has a continuous thread that extends from the proximal end 52 of the third zone 36 to the distal end 54 of the first zone 32. The first and third zones 32, 36 having threads 44 having a first distance d measured between the crests of two adjacent threads 44, wherein the threads 44 of the second zone 34 have a second distance d¹ that is a multiple of distance d. In one embodiment in accordance with the present disclosure the second distance d¹ of the second zone 34 is twice the first distance d of the first and third zones 32, 36. It is also envisioned that the bone fastener 30 of the present disclosure can have threads in each of the three zones that have different distances such that two of the zones have a distance that is a multiple of the smaller distance and different than the other zone.

The threads in the first and third zones 32, 36 being closer together improve fixation in dense tissue, such as cortical bone, while threads in the second zone 34 having a distance between threads that is approximately twice that of the distance between threads in first and third zones 32, 36, improve fixation in tissue that is less dense, such as cancellous bone. Accordingly, the first zone 32 and third zone 36, which are configured to be fixed into cortical bone, have approximately twice as many threads along a given length of the elongated shaft, while the second zone 34, which is configured to be fixed into cancellous bone, has fewer threads, therefore, more bone between each thread so as to securely set the bone fastener into cancellous bone.

As shown in FIGS. 1 and 2, first zone 32 has about six threads, second zone 34 has about nine threads and third zone 36 has about six threads. However, it is envisioned, for example, that the first zone 32 may have between about five and twelve threads, second zone 34 may have between eight and 12 threads and the third zone 36 may have between five and twelve threads. It is also envisioned that there can be three or more zones and the zones may have the same number of threads or different numbers of threads depending on the type of bone the fastener is to be inserted into.

In one embodiment, the outer diameter of the shaft of bone fastener 30 tapers from proximal end 56 of first zone 32 to distal end 54 of first zone 32 such that first zone 32 is conical, and the outer diameter of the shaft of bone fastener 30 is uniform along the length of second and third zones 34, 36 such that second and third zones 34, 36 are cylindrical. In one embodiment, the outer diameter of the shaft of bone fastener 30 tapers along the length of the first and second zones from proximal end 48 of second zone 34 to distal end 54 of first zone 32 such that first and second zones 32, 34 define a conical section of the shaft, and the outer diameter of the shaft of bone fastener 30 is constant along the length of third zone 36 such that third zone 36 defines a cylindrical portion of the shaft. In one embodiment, the outer diameter of the shaft of bone fastener 30 tapers along the length of first, second and third zones 32, 34, 36 from proximal end 52 of third zone 36 to distal end 54 of first zone 32 such that first, second and third zones 32, 34, 36 define a conical section of the shaft. In one embodiment, the outer diameter of first, second and third zones 32, 34, 36 is uniform such that first, second and third zones 32, 34, 36 are cylindrical.

Proximal end 52 of third zone 36 has a head 58. In one embodiment, head 58 has extending proximally therefrom configured to engage a tool to facilitate fixation of bone fastener 30 into bone or other tissue. In one embodiment head 60 is configured to accept a tool, such as a screwdriver. However, it is also envisioned that head 58 may have any configuration suitable to engage a tool designed to rotate bone fastener 30 so as to fix bone fastener 30 within bone or other tissue. It is also envisioned that any attachment method to a longitudinal rod can be used. Head 58, in one embodiment, has a diameter at a distal end 60 that is approximately equal to or greater than the diameter of lead portion 32, intermediate portion 34 and/or tail portion 36. However, it is envisioned that the diameter of proximal end 60 of head 58 may also be less than the diameter of lead portion 32, intermediate portion 34 and/or tail portion 36. Head 58 may have a different cross-sectional area, geometry, material or material property. In one embodiment, head 58 may be threaded to receive an implant, such as extender. Bone fastener 30 includes a transition portion 62 between proximal end 52 of third zone 36 and head 58. Transition portion 62 may be tapered or cylindrical. In one embodiment, head 58 has a diameter that is less than the diameter of thread 44 at proximal end 52 of the third zone 36 and/or the proximal end 60 of head 58. The diameter of bone fastener 30 decreases gradually between thread 44 at proximal end 52 of the third zone 36 and transition portion 62 and between proximal end 60 of head 58 and transition portion 62. That is, the diameter of thread 44 at proximal end 52 of third zone 36 and proximal end 60 of head 58 are both greater than the diameter of transition portion 62. In one embodiment, head 58 may be threaded to receive an implant.

Bone fastener 30 is configured to be fixed within bone having distinct regions with differing characteristics such as bone density. Accordingly, bone fastener 30 has portions or zones with characteristics that correspond to the characteristics of the region of the bone they are being inserted within so as to provide secure fixation within each region of bone. As discussed above, first zone 32 and third zone 36 are configured to be fixed within cortical bone 100 and second zone 34 is configured to be fixed within cancellous bone 102. That is, bone fastener 30 is configured to be inserted within bone having sections of cortical bone 100 positioned between cancellous bone 102 such that when bone fastener 30 is inserted into a bone, first zone 32 and third zone 36 lie within cortical bone 100 and second zone 34 lies within cancellous bone 102, as shown in FIG. 2. The threads of first zone 32 can be configured so as to be self-tapping. It is envisioned that the angle between each thread along the elongated shaft of the first, second and third zones 32, 34, 36 is constant. It is also envisioned that the helix angle of thread 44 along the length of first zone 32 may be different from the helix angle of thread 44 along the length of third zones 36. It is contemplated that thread 42 along the length of second zone 34 can be a single lead thread, while thread 44 along the length of first and third zones 32, 36 can be a dual, triple or quadruple lead thread. Further, the lead of thread 42 along the length of second zone 34 should be the same as the lead of thread 44 along the length of first and third zones 32, 36.

In one embodiment, in accordance with the principles of the present disclosure, thread 44 along the length of first zone 32 is configured to create a pair of helical grooves as it is advanced into the bone. The internal helical grooves being defined by a series of voids connected together by a helical configuration along the length of the first zone 32. As second zone 34 is advanced into the internal helical groove, every other void in at least a portion of the internal helical groove receives thread 42. In embodiments in which second zone 34 has a diameter which is greater than that of first zone 32, the shaft of second zone 34 fills the void created by thread 44 along the length of first zone 32 and creates a helical groove in the bone with a greater diameter than the diameter of the threads in the first zone 32. The voids in the cancellous bone that are not filled with thread 42 of second zone 34 are at least partially covered by and/or abuts against the shaft of bone fastener 30 that is free of threads. Covering the voids in the cancellous bone not only prevents infection, but also strengthens the connection to the naturally porous, less dense cancellous bone. As third zone 36 is advanced into the internal helical groove, each void in the internal helical groove receives thread 42 along the length of third zone 36 when bone fastener 30 is positioned in bone. The second thread 44 in the third zone 36 cuts a lead in the bone as the screw is advanced into the cortical bone 100. In embodiments in which third zone 36 has a diameter which is greater than that of second zone 34, thread 44 along the length of third zone 36 will increase the depth of each void of the internal helical groove before filling those voids as third zone 36 is advanced into the internal helical groove.

In use, the bone fastener 30, in accordance with the principles of the present disclosure is affixed into a bone having cancellous bone between a first and second layer of cortical bone, such as the pelvis. A method of fixation to a bony body is provided, which includes the steps of exposing a surface of the bony body to receive a bone fastener and threading the self-tapping lead portion of the bone fastener into the bony body. The self-tapping lead portion of the bone fastener 30 is advanced by rotating the bone fastener to cut a continuous helical groove into the first layer of cortical bone, followed by the cancellous portion of bone and finally into the second layer of cortical bone. When set in a bone having cancellous bone between a first and second layer of cortical bone, such as the pelvis, the first zone will set in the first layer of cortical bone, the second zone will set in the cancellous layer of bone and the third zone will set in the second layer of cortical bone, thereby securing the bone fastener into bone. In one embodiment, the method further includes drilling a pilot hole to facilitate insertion of bone fastener 30. It is understood that the diameter of the pilot hole should be less than or equal to the outer diameter of first zone 32. It is envisioned that the pilot hole may be either cylindrical or tapered.

It is contemplated that bone fastener 30 may be coated with an osteoconductive material such as hydroxyapatite and/or osteoinductive agent such as a bone morphogenic protein for enhanced bony fixation. Bone fastener 30 can be made of radiolucent materials such as polymers. Radiomarkers may be included for identification under x-ray, fluoroscopy, CT or other imaging techniques. Metallic or ceramic radiomarkers, such as tantalum beads, tantalum pins, titanium pins, titanium endcaps and platinum wires can be used.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A bone fastener comprising:
a head, and
an elongated shaft with a leading edge;
wherein said elongated shaft has a first zone, a second zone and a third zone, and the second zone is disposed between the first zone and the third zone; and
wherein the shaft has a first continuous helical thread along the length of the first zone, the second zone and the third zone and a second helical thread extending along the length of the first zone and the third zone, the second zone being devoid of the second helical thread.

2. The bone fastener of claim 1, wherein the thread along the length of the first zone is configured to create an internal helical groove in bone, the internal helical groove being defined by a series of voids connected together by a helical configuration along the length of the internal helical groove such that every other void in at least part of the internal helical groove receives the threads along the length of the second zone when the bone fastener is positioned in bone.

3. The bone fastener of claim 2, wherein the voids along the internal helical grooves formed by the first zone that do not receive the threads of the second zone are at least partially covered with a portion of the elongated shaft between threads in the second zone.

4. The bone fastener of claim 1, wherein the thread of the first zone is self-tapping.

5. The bone fastener of claim 1, wherein the outer diameter of the shaft along the length of the first zone tapers from a proximal end of the first zone to a distal end of the first zone such that the first zone is conical; and the outer diameter of the shaft along the length of the second and third zones is uniform such that the second and third zones are cylindrical.

6. The bone fastener of claim 1, wherein the outer diameter of the first, second and third zones is uniform such that the first, second and third zones are cylindrical.

7. The bone fastener of claim 1, wherein the pitch of the threads in the second zone is approximately twice the pitch of the threads in the first and third zones.

8. The bone fastener of claim 1, wherein the helical angle between each thread along the elongated shaft of the first, second and third zones is constant.

9. The bone fastener of claim 1, wherein the threads along the length of the first and third zones are dual lead threads and the threads along the length of the second zone are single lead threads.

10. The bone fastener of claim 1, wherein the outer diameter of the shaft along the length of the first and second zones tapers from a proximal end of the second zone to a distal end of the first zone such that the first and second zones define a conical section of the shaft; and the outer diameter of the shaft along the length of the third zone is constant such that the third zone defines a cylindrical portion of the shaft.

11. A bone fastener for anchoring into a bone of a patient having cancellous bone between a first and second layer of cortical bone comprising:
a head, and
an elongated shaft with a leading edge;
wherein said shaft has a first zone, a second zone and a third zone, and the second zone is disposed between the first zone and the third zone;
wherein the shaft has a continuous helical groove along the length of the first zone, the second zone and the third zone;
wherein a distance between threads in the second zone is approximately twice a distance between threads in the first and second zones;
wherein the bone fastener is configured such that the first zone and the third zone engage cortical bone and the second zone engages cancellous bone when the bone fastener is fully set within the bone of the patient.

12. The bone fastener of claim 11, wherein the thread along the length of the first zone is configured to create an internal helical groove in the first and second cortical bone layers and the cancellous bone layer, the internal helical groove being defined by a series of voids connected together by a helical configuration along the length of the internal helical groove such that a portion of the voids in the internal helical groove in the cancellous bone layer is configured to receive the threads along the length of the second zone when the bone fastener is fully set in the bone of the patient.

13. The bone fastener of claim 11, wherein the thread of the first zone is self-tapping.

14. The bone fastener of claim 11, wherein the outer diameter of the shaft along the length of the first zone tapers from a proximal end of the first zone to a distal end of the first zone such that the first zone is conical; and the outer diameter of the shaft along the length of the second and third zones is uniform such that the second and third zones are cylindrical.

15. The bone fastener of claim 14, wherein the outer diameter of the shaft along the length of the first and second zones tapers from a proximal end of the second zone to a distal end of the first zone such that the first and second zones define a conical section of the shaft; and the outer diameter of the shaft along the length of the third zone is constant such that the third zone defines a cylindrical portion of the shaft.

16. The bone fastener of claim 11, wherein the thickness of the threads and the angle between each thread along the elongated shaft of the first, second and third zones are constant.

17. The bone fastener of claim 11, wherein the thread along the length of the first and third zones is a dual lead thread and the thread along the length of the second zone is a single lead thread.

18. The bone fastener of claim 12, wherein an inner diameter of the second zone is greater than an outer diameter of the first zone such that a thread free region of the second zone at least partially covers the voids along the length of the internal thread not filled by threads such that every other void in at least a portion of the internal helical groove in the cancellous bone is either filled by the threads of the second zone or at least partially covered by the thread free region of the second zone.

19. The bone fastener of claim 11, wherein the outer diameter of the first, second and third zones is uniform such that the first, second and third zones are cylindrical.

20. A method for anchoring a bone fastener into a bone of a patient having cancellous bone between a first and second layer of cortical bone comprising:
i) exposing a surface of a bony body to receive a bone fastener having a head and an elongated shaft with a self-tapping leading edge, wherein the elongated shaft has a continuous helical thread along the length of a first zone, a second zone and a third zone wherein a distance between threads in the second zone is approximately twice the distance between threads in the first and second zones;
ii) advancing said bone fastener into the bony body to form a helical groove through the first and second layers of cortical bone as well as the cancellous layer between the first and second layers of cortical bone; and
iii) setting said bone fastener into the bony body such that the first and third zones set into the first and second layers of cortical bone and second zone set into cancellous bone.

21. The bone fastener of claim 1, wherein the outer diameter of the shaft along the length of the first, second and third zones tapers from a proximal end of the third zone to a distal end of the first zone such that the first, second and third zones define a conical section of the shaft.

22. The bone fastener of claim 11, wherein the outer diameter of the shaft along the length of the first, second and third zones tapers from a proximal end of the third zone to a distal end of the first zone such that the first, second and third zones define a conical section of the shaft.
